(19) United States
(12) Patent Application Publication
(10) Pub. No.: US 2008/0275095 A1
(43) Pub. Date: Nov. 6, 2008

(54) Combination of Prostaglandin E2 Receptor Antagonists and Renin-Angiotensin System Inhibitors for Treating Renal Diseases

(75) Inventors: Hajime Sogabe, Tokyo (JP); Masayuki Tomita, Tokyo (JP); Shoko Nakazato, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Astellas Pharma Inc., Tokyo (JP)

(21) Appl. No.: 11/579,507

(22) PCT Filed: Mar. 28, 2005
(86) PCT No.: PCT/JP2005/006536

§ 371 Date: Jan. 29, 2007

Related U.S. Application Data
(60) Provisional application No. 60/566,920, filed on May 3, 2004.

(51) Int. Cl. A61K 31/421; A61P 13/12

(52) U.S. Cl. 514/374

(57) ABSTRACT

This invention relates to methods for treating renal diseases by using combination of prostaglandin E2 receptor antagonist and renin-angiotensin system inhibitor and pharmaceutical compositions for treating renal diseases comprising prostaglandin E2 receptor antagonist and renin-angiotensin system inhibitor.

### TECHNICAL FIELD

This invention relates to the combination of prostaglandin E2 receptor antagonist and renin-angiotensin system inhibitor for treating renal diseases.

More particularly, this invention relates to methods for treating renal diseases by using the combination of prostaglandin E2 receptor EP4 antagonist and renin-angiotensin system inhibitor and pharmaceutical compositions for treating renal diseases comprising prostaglandin E2 receptor EP4 antagonist and renin-angiotensin system inhibitor.

### BACKGROUND ART

Prostaglandin E2 is known as one of the metabolites in an arachidonate cascade. It is known that it has various activities such as pain inducing activity, inflammatory activity, uterine contractile activity, a promoting effect on digestive peristalsis, an awaking activity, a suppressive effect on gastric acid secretion, hypotensive activity, platelet inhibitory activity, bone-resorbing activity, angiogenic activity, or the like.

Prostaglandin E2 receptors have been sub-divided into four subtypes, EP1, EP2, EP3 and EP4, and these receptors have a wide distribution in various tissues. The effects associated with EP1 and EP3 receptors may be considered as excitatory, and are believed to be mediated by stimulation of phosphatidylinositol turnover or inhibition of adenylcyclase activity, with resulting decrease in intracellular levels of cyclic AMP. In contrast, the effects associated with EP2 and EP4 receptors may be considered as inhibitory, and are believed to be associated with a stimulation of adenylcyclase and an increase in levels of intracellular cyclic AMP.

EP4 receptor is highly expressed in thymus, intestine, lung, spleen, adrenal grand, kidney, and so on, it may be considered to be associated with smooth muscle relaxation, anti-inflammatory or pro-inflammatory activities, lymphocyte differentiation, antiallergic activities, mesangial cell relaxation or proliferation, gastric or enteric mucus secretion, or the like. In kidney, EP4 receptor is predominantly expressed in glomerulus, where it may contribute to the regulation of glomerular hemodynamics and renin release. To date, a variety of EP4 antagonists have been developed for treating renal diseases, and it has been shown that some of EP4 agonists may also have therapeutic effect on said diseases. However, the mechanism by which EP4 antagonist or agonist can function for treating renal diseases have not been elucidated.

Angiotensin I (AI) is, in the renin-angiotensin cascade, generated from angiotensinogen in blood plasma by the enzymatic action of renin produced in kidney. Then, angiotensin I is converted to angiotensin II (AII) by angiotensin converting enzyme (ACE). Angiotensin II has a potent vasoconstrictive activity and therefore can cause hypertension. Therefore, angiotensin converting enzyme inhibitors (ACE-I), which inhibit the production of angiotensin II, and angiotensin II receptor blocker (ARB), which inhibit the function of angiotensin II (collectively referred to renin-angiotensin system inhibitors) have been used in the treatment of hypertension.

Renin-angiotensin system inhibitors have been also used in the treatment of nephritis or renal failure. In renal failure, Na is pooled in the blood to raise blood pressure, and angiotensin II contracts efferent glomerular arteriole to raise intraglomerular pressure. The rise of blood pressure and intraglomerular pressure cause and maintain glomerular hyperfiltration to promote progression of renal failure pathologically. Therefore, renin-angiotensin system inhibitors can be used to suppress intraglomerular hypertension to prevent the progression of renal failure pathologically. Renin-angiotensin system inhibitors specifically relax efferent glomerular arteriole and can suppress intraglomerular hypertension independent on systemic blood pressure. However, the efficacy of renin-angiotensin system inhibitors in the treatment of renal disease is not sufficient and can not increase by administering higher amounts of the inhibitors.

### SUMMARY OF THE INVENTION

The present inventors have established use of the combination of prostaglandin E2 receptor antagonist and renin-angiotensin system inhibitor in the treatment of renal failure to overcome the low efficacy of the prostaglandin E2 receptor antagonist or the renin-angiotensin system inhibitor alone in such treatment.

Specifically, the present invention is based on the knowledge found by the inventors that EP4 receptor antagonists can function in the treatment of renal failure by a mechanism different from that of renin-angiotensin system inhibitors and the combination of EP4 receptor antagonists and renin-angiotensin system inhibitors, rather than EP4 receptor antagonists or renin-angiotensin system inhibitors alone, can more effectively suppress the rate of decline in renal function.

In one aspect, the present invention relates to methods for treating renal diseases comprising administering prostaglandin E2 receptor antagonist and renin-angiotensin system inhibitor to a patient suffering from said diseases and pharmaceutical compositions for treating renal, diseases comprising prostaglandin E2 receptor antagonist and renin-angiotensin system inhibitor.

In another aspect, the present invention relates method for increasing an effect of renin-angiotensin system inhibitor for treating renal disease, comprising administering prostaglandin E2 receptor antagonist to a patient suffering from said disease, and pharmaceutical composition for increasing an effect of renin-angiotensin system inhibitor for treating renal disease, comprising prostaglandin E2 receptor antagonist.

In another aspect, the present invention relates method for increasing an effect of prostaglandin E2 receptor antagonist for treating renal disease, comprising administering renin-angiotensin system inhibitor to a patient suffering from said disease, and pharmaceutical composition for increasing an effect of prostaglandin E2 receptor antagonist for treating renal disease, comprising renin-angiotensin system inhibitor.

In another aspect, the present invention relates to a use of prostaglandin E2 receptor antagonist and renin-angiotensin system inhibitor for manufacturing a medicament for treating renal disease.

In another aspect, the present invention relates to a use of prostaglandin E2 receptor antagonist for manufacturing a medicament for increasing an effect of renin-angiotensin system inhibitor for treating renal disease, and a use of renin-angiotensin system inhibitor for manufacturing a medicament for increasing an effect of prostaglandin E2 receptor antagonist for treating renal disease.

In another aspect, the present invention relates to an article of manufacture, comprising packaging material and prostaglandin E2 receptor antagonist within the packaging material, wherein said packaging material comprises a label or a written material which indicates that the prostaglandin E2 receptor antagonist can be used for increasing an effect of renin-angiotensin system inhibitor for treating renal disease.

In another aspect, the present invention relates to an article of manufacture, comprising packaging material and renin-angiotensin system inhibitor within the packaging material, wherein said packaging material comprises a label or a written material which indicates that the renin-angiotensin system inhibitor can be used for increasing an effect of prostaglandin E2 receptor antagonist for treating renal disease.

### BRIEF DESCRIPTION OF THE DRAWINGS

FIG. 1 shows the time schedule in the experiment of the Example using male Wistar rats.

FIG. 2 shows the influence of the combination of the present invention on body weight gain.

FIG. 3 shows the effect of the combination of the present invention on urinary excretion of the protein.

FIG. 4 shows the effect of the combination of the present invention on blood urea nitrogen (BUN).

FIG. 5 shows the effect of the combination of the present invention on the serum creatinine concentration.

FIG. 6 shows the effect of the combination of the present invention on the percentage of rats reached endpoint.

FIG. 7 shows the effect of the combination of the present invention on the rate of decline in renal function.

FIG. 8 shows the prediction of time reaching renal death.

FIG. 9 shows the effect of the combination of the present invention on systolic blood pressure.

FIG. 10 shows the effect of the combination of the present invention on kidney weight.

FIG. 11 shows the effect of the combination of the present invention on the number of red blood cell.

FIG. 12 shows histological findings.

FIG. 13 shows the effect of the combination of the present invention on glomerular injury.

### DETAILED DESCRIPTION OF THE PRESENT INVENTION

The present invention is now described in detail.

Prostaglandin E2 receptor antagonists useful for the present invention include a variety of prostaglandin E2 receptor antagonists known in the art.

Preferably, prostaglandin E2 receptor antagonist useful for the present invention is an EP4 receptor antagonist.

Preferably, EP4 receptor antagonists useful for present invention include known oxazole compounds described in, for example, WO95/17393, WO95/24393, WO97/03973, WO98/55468 and WO00/18744, and compounds described in WO00/03980, WO0/15608, WO00/18405, WO00/21532, WO01/10426, WO01/49661, WO01/72302, WO01/42281, WO01/62708, WO02/16311, WO02/20462, WO02/32900, WO03/086390, WO03/087061, WO02/50031, WO02/50032, WO02/50033, WO02/64564, which are incorporated by reference in the present specification, or pharmaceutically acceptable salts thereof.

Preferably, EP4 receptor antagonists useful for present invention include the oxazole compounds represented by the following formula (I):

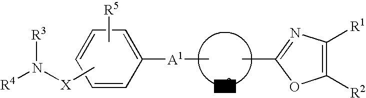

wherein

R¹ is aryl which may be substituted with halogen(s),

R² is aryl which may be substituted with halogen(s),

X is single bond,

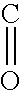

or SO₂,

R³ and R⁴ are independently hydrogen or suitable substituent

(wherein X is

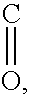

neither R³ nor R⁴ is hydrogen),

R³ and R⁴ may be linked together to form

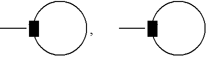

is N-containing heterocyclic group which may be substituted with one or more suitable substituent(s),
R⁵ is hydrogen, hydroxyl, carboxy, or protected carboxy,
A¹ is lower alkylene or single bond,

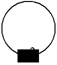

is cyclo(C₃-C₉) alkane or cyclo(C₅-C₉)alkene,
or a pro-drug thereof, or a pharmaceutical acceptable salt thereof.

The definitions used in the above general formula (I) and the specific and preferred examples thereof are explained and set forth in detail as follows.

Suitable “aryl” and aryl moiety in the terms “ar(lower) alkyl”, “aryloxy”, “ar(lower)alkenyl”, “arylsulfonyl”, “ar(lower) arylsulfonyl”, “ar(lower)alkylsulfonyl”, and “aryl oxysulfonyl” may include phenyl, lower alkyl phenyl (e.g., tolyl, ethylphenyl, propylphenyl, etc.), naphthyl or the like.

Suitable “halogen” may include fluorine, chlorine, bromine, or iodine.

The term “lower” is intended to mean 1 to 6 carbon atom(s), unless otherwise indicated.

Suitable “lower alkyl” and lower alkyl moiety in the terms “lower alkylamino”, “ar(lower)alkyl”, “carboxy(lower)alkyl”, “hydroxy(lower)alkyl”, “ar(lower)alkylsulfonyl”, and lower alkylsulfonyl may include straight or branched one having 1 to 6 carbon atom(s), such as methyl, ethyl, propyl, isopropyl, butyl, isobutyl, sec-butyl, t-butyl, pentyl, t-pentyl, hexyl or the like, preferably one having 1 to 4 carbon atom(s).

Suitable “lower alkylamino” may include mono- or di-(lower) alkylamino, such as methylamino, dimethylamino, ethylamino, diethylamino, or the like.

Suitable “lower alkoxy” and lower alkoxy moiety in the term “hydroxy(lower)alkoxy” may include methoxy, ethoxy propoxy, isopropoxy, butoxy, isobutoxy, t-butoxy, pentyloxy, t-pentyloxy, hexyloxy, or the like, preferably methoxy.

Suitable “heterocyclic group” may include saturated or unsaturated, monocyclic or polycyclic heterocyclic group containing at least one nitrogen atom. And especially preferable heterocyclic ring containing nitrogen may be ones such as:
unsaturated 3 to 8-membered heteromonocyclic group containing 1 to 4 nitrogen atom(s), for example, pyrrolyl, pyrrolinyl, imidazolyl, pyrazolyl, pyridyl and its N-oxide, pyrimidinyl, pyrazinyl, dihydropyridazinyl, tetrahydropyridazinyl, triazolyl (e.g., 1H-1,2,4-triazolyl, 1H-1,2,3-triazolyl, 2H-1,2,3-triazolyl, etc.), tetrazolyl (e.g., 1H-tetrazolyl, 2H-tetrazolyl, etc.), dihydrotriazinyl (e.g., 4,5-dihydro-1,2,4-triazinyl, 2,5-dihydro-1,2,4-triazinyl, etc.),etc., saturated 3 to 8-membered heteromonocyclic group containing 1 to 4 nitrogen atom(s), for example, pyrrolidinyl, imidazolidinyl, piperidinyl, piperazinyl, azacycloheptyl, azacyclooctyl, perhydroazepinyl, etc.,
unsaturated condensed heterocyclic group containing 1 to 5 nitrogen atom(s), for example, indolyl, 2,3-dihydroindolyl, isoindolyl, indolinyl, indazolyl, isoindolinyl, indolizinyl, benzimidazolyl, quinolyl, 1,2,3,4-tetrahydroquinolyl, isoquinolyl, indazolyl, benzotriazolyl, tetrazolopyridyl, tetrazolopyridazinyl (e.g., tetrazolo[1,5-b]pyridazinyl, etc.,), dihydrotriazolopyridazinyl, etc.,
unsaturated 3 to 8-membered heteromonocyclic group containing 1 to 2 oxygen atom(s) and 1 to 3 nitrogen atom(s), for example, oxazolyl, isoxazolyl, dihydroisoxazolyl, oxadiazolyl (e.g., 1,2,4-oxadiazolyl, 1,3,4-oxadiazolyl, 2,5-oxadiazolyl, etc.,), etc.,
saturated 3 to 8-membered heteromonocyclic group containing 1 to 2 oxygen atom(s) and 1 to 3 nitrogen atom(s), for example, morpholino, etc.,
unsaturated condensed heterocyclic group containing 1 to 2 oxygen atom(s) and 1 to 3 nitrogen atom(s), for example, benzoxazolyl, benzoxadiazolyl, etc.,
unsaturated 3 to 8-membered heteromonocyclic group containing 1 to 2 sulfur atom(s), for example, thienyl, thiepinyl, etc.,
unsaturated 3 to 8-membered heteromonocyclic group containing 1 to 2 sulfur atom(s) and 1 to 3 nitrogen atom(s), for example, thiazolyl, isothiazolyl, thiazolinyl, thiadiazolyl (e.g., 1,2,4-thiadiazolyl, 1,3,4-thiadiazolyl, 1,2,5-thiadiazolyl, 1,2,3-thiadiazolyl), etc.,
saturated 3 to 8-membered heteromonocyclic group containing 1 to 2 sulfur atom(s) and 1 to 3 nitrogen atom(s), for example, thiazolidinyl, etc.,
unsaturated condensed heteromonocyclic group containing 1 to 2 sulfur atom(s) and 1 to 3 nitrogen atom(s), for example, benzothiazolyl, benzothiadiazolyl, etc., and the like.

Suitable acyl and acyl moiety in the terms of “acylamino” and “acyloxy” may include aliphatic acyl group and acyl group containing an aromatic or heterocyclic ring.

And, suitable examples of the said acyl may be lower alkanoyl (e.g. formyl, acetyl, propionyl, butyryl, isobutyryl, valeryl, isovaleryl, oxalyl, succinyl, pivaloyl, etc.); lower alkenoyl (e.g., propionyl, 2-methylpropionyl, butenoyl, or the like, preferably one having 3 to 4 carbon atom(s)); aroyl (benzoyl, naphthoyl, etc.); lower alkoxyaroyl (methoxyphenylcarbonyl, ethoxyphenylcarbonyl, propoxyphenylcarbonyl, isopropoxyphenylcabonyl, methoxynaphthylcarbonyl, ethoxynaphthylcarbonyl, propoxynaphtylcarbonyl, isopropoxynaphthylcarbonyl, etc.); heterocyclic carbonyl (“heterocyclic moiety” in the term “heterocyclic carbonyl” can be referred above); bridged cyclic(lower)alkanecarbonyl(bicyclo[2.2.1]hept-2-yl-carbonyl, bicyclo[3.2.1]oct-2-yl-carbonyl, bicyclo[3.2.2]non-2-yl-carbonyl, bicyclo[3.2.2]non-3-yl-carbonyl, bicyclo[4.3.2]undec-2-yl-carbonyl, bicyclo[4.3.2]undec-3-yl-carbonyl, bicyclo[2.2.2]oct-2-en-2-yl-carbonyl, bicyclo[3.2.2]non-3-en-3-yl-carbonyl, tricyclo[5.3.1.1]dodec-2-yl-carbonyl, tricyclo[5.3.1.1]dodec-3-yl-carbonyl, adamantylcarbonyl, etc.); cyclo(lower)-alkanecarbonyl (cyclopropanecarbonyl, cyclobutanecarbonyl, cyclopentanecarbonyl, cyclohexane-carbonyl, etc.), carbamoyl which may be substituted with mono- or di-(lower) alkyl (e.g. dimethylcarbamoyl, etc.) and the like.

Suitable “cyclo(lower)alkyl” may include cyclopropyl, cyclopentyl, cyclobutyl, cyclohexyl, or the like.

Suitable “cyclo(lower) alkenyl” may include cyclopentenyl, cyclohexenyl, cycloheptenyl, cyclooctenyl, cyclononenyl, or the like.

Suitable “protected carboxy” may include carboxylate, esterified carboxy, or the like.

Suitable example of the ester moiety of an esterified carboxy may be the ones such as lower alkyl (e.g., methyl, ethyl, propyl, isopropyl, butyl, isobutyl, tert-butyl, pentyl, hexyl, etc.) which may have at least one suitable substituent (s), for example, lower alkanoyloxy(lower)alkyl (e.g., acetoxymethyl, butyryloxymethyl, valeryloxymethyl, pivaloyloxymethyl, etc.), halo(lower)alkyl (e.g., 2-iodoethyl, 2,2,2-trichloroethyl, etc.); lower alkenyl (e.g., vinyl, allyl, etc.); lower alkynyl (e.g., ethynyl, propynyl, etc.); ar(lower)alkyl which may have at least one suitable substituent(s) (e.g., benzyl, 4-methoxybenzyl, 4-nitrobenzyl, phenethyl, trityl, etc.); aryl which may have at least one suitable substituent(s) (e.g., phenyl, tolyl, 4-chlorophenyl, tert-butylphenyl, xylyl, mesityl, cumenyl, etc.); phthalidyl; or the like.

Suitable “lower alkylene” may include straight or branched one having 1 to 6 carbon atom(s), such as methylene, ethylene, trimethylene, tetramethylene, pentamethylene and hexamethylene, preferably one having 1 to 3 carbon atom(s), more preferably methylene.

Suitable “cyclo(C₃-C₉)alkane” may include cyclopropane, cyclobutane, cyclopentane, cyclohexane, cycloheptane, cyclooctane, cyclononane, or the like, preferably one having 5 to 7 carbon atoms.

Suitable “cyclo(C₅-C₉)alkene” may include cyclopentene, cyclohexene, cycloheptene, cyclooctene, cyclononene, or the like, preferably one having 5 to 7 carbon atoms.

Preferred embodiments of the oxazole compounds (I) as EP4 receptor antagonists useful for the present invention are as follows:

wherein

R¹ is aryl which may be substituted with halogen(s),

R² is aryl which may be substituted with halogen(s),

X is single bond,

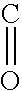

or SO₂,

R³ and R⁴ are independently
(1) hydrogen;
(2) hydroxy;
(3) lower alkyl which may be substituted with one or more substituent(s) selected from the group consisting of:
   (a) hydroxy,
   (b) cyano,
   (c) lower alkoxy,
   (d) hydroxy(lower)alkoxy,
   (e) cyclo(lower)alkyl,
   (f) cyclo(lower)alkenyl,
   (g) amino,
   (h) lower alkylamino,
   (i) carbamoyl,
   (j) carboxy,
   (k) protected carboxy,
   (l) heterocyclic group optionally substituted with ar(lower)alkyl or oxo, and
   (m) aryl optionally substituted with
      hydroxy,
      carboxy,
      protected carboxy,
      carboxy(lower)alkyl, or
      lower alkoxy which may be substituted with carboxy or protected carboxy;
(4) lower alkoxy which may be substituted with aryl(s);
(5) aryl which may be substituted with one or more substituent (s) selected from the group consisting of:
   (a) aryloxy,
   (b) acylamino, and
   (c) carbamoyl;
(6) cyclo(lower)alkyl which may be substituted with hydroxy(s);
(7) arylsulfonyl;
(8) ar(lower)alkylsulfonyl;
(9) lower alkylsulfonyl;
(10) aryloxysulfonyl;
(11) heterocyclic group which may be substituted with one or more substituent(s) selected from the group consisting of:
   (a) ar(lower)alkyl,
   (b) aryl,
   (c) protected carboxy,
   (d) lower alkyl, and
   (e) oxo;
(12) acyl which may be substituted with aryl; or
(13) carbamoyl which may be substituted with acyl, ar(lower)alkyl, or arylsulfonyl,

(wherein X is

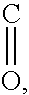

neither R³ nor R⁴ is hydrogen),

R³ and R⁴ may be linked together to form

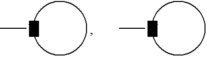

is N-containing heterocyclic group which may be substituted with one or more substituent(s) selected from the group consisting of:
(1) lower alkyl,
(2) aryl,
(3) protected carboxy,
(4) hydroxy(lower)alkyl,
(5) ar(lower)alkyl,
(6) hydroxy,
(7) oxo, and
(8) lower alkylamino,

R⁵ is
(1) hydrogen,
(2) hydroxy,
(3) carboxy, or
(4) protected carboxy,

A¹ is lower alkylene or single bond,

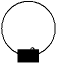

is cyclo (C₃-C₉) alkane or cyclo (C₅-C₉) alkene,

or a pro-drug thereof, or a pharmaceutically acceptable salt thereof.

More preferred embodiments of the oxazole compounds (I) as EP4 receptor antagonists useful for the present invention are as follows:

wherein

R¹ is aryl,

R² is aryl,

X is single bond

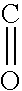

or SO₂,

R³ and R⁴ are independently
(1) hydrogen;
(2) hydroxy;
(3) lower alkyl which may be substituted with one or more substituent(s) selected from the group consisting of:
   (a) hydroxy,
   (b) cyano,
   (c) lower alkoxy,
   (d) hydroxy(lower)alkoxy,
   (e) cyclo(lower)alkyl,
   (f) cyclo(lower)alkenyl,
   (g) amino,
   (h) lower alkylamino,
   (i) carbamoyl,
   (j) carboxy,
   (k) protected carboxy,
   (l) heterocyclic group optionally substituted with ar(lower)alkyl or oxo, and
   (m) aryl optionally substituted with
      hydroxy,
      carboxy,
      protected carboxy,
      carboxy(lower)alkyl, or
      lower alkoxy which may be substituted with carboxy or protected carboxy;
(4) lower alkoxy which may be substituted with aryl(s);
(5) aryl which may be substituted with one or more substituent (s) selected from the group consisting of:
   (a) aryloxy,
   (b) acylamino, and
   (c) carbamoyl;
(6) cyclo(lower)alkyl which may be substituted with hydroxy(s);
(7) arylsulfonyl;
(8) ar(lower)alkylsulfonyl;
(9) lower alkylsulfonyl;
(10) aryloxysulfonyl;
(11) heterocyclic group which may be substituted with one or more substituent(s) selected from the group consisting of:
   (a) ar(lower)alkyl,
   (b) aryl,
   (c) protected carboxy,
   (d) lower alkyl, and
   (e) oxo;
(12) acyl which may be substituted with aryl; or
(13) carbamoyl which may be substituted with acyl, ar(lower)alkyl, or arylsulfonyl,

(wherein X is

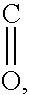

neither R³ nor R⁴ is hydrogen),

R³ and R⁴ may be linked together to form

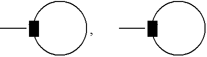

is N-containing heterocyclic group which may be substituted with one or more substituent(s) selected from the group consisting of:
(1) lower alkyl,
(2) aryl,
(3) protected carboxy,
(4) hydroxy(lower)alkyl,
(5) ar(lower)alkyl,
(6) hydroxy,
(7) oxo, and
(8) lower alkylamino,

R⁵ is hydrogen,

A¹ is lower alkylene,

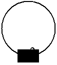

is
(1) cyclohexane,
(2) cyclohexene,
(3) cyclopentane, or
(4) cyclopentene,

or a pro-drug thereof, or a pharmaceutically acceptable salt thereof.

Furthermore preferred embodiments of the oxazole compounds (I) are as follows

wherein

R¹ is phenyl,

R² is phenyl,

X is

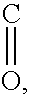

or SO₂,

R³ and R⁴ are independently
(1) hydrogen;
(2) lower alkyl which may be substituted with one or more substituent(s) selected from the group consisting of:
   (a) hydroxy,
   (b) heterocyclic group, and
   (c) phenyl;
(3) lower alkoxy which may be substituted with phenyl; or
(4) phenyl which may be substituted with phenyloxy;

(wherein X is

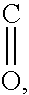

neither R³ nor R⁴ is hydrogen),

R³ and R⁴ are linked together to form

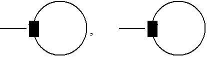

is N-containing heterocyclic group;

R⁵ is hydrogen,

A¹ is methylene,

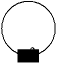

is
(1) cyclohexane,
(2) cyclohexene,
(3) cyclopentane, or
(4) cyclopentene,
   or a pro-drug thereof, or a pharmaceutically acceptable salt thereof.

The specifically preferred EP4 receptor antagonists useful for the present invention is N-[(2-hydroxy-2-phenyl)ethyl]-3-{[(1S,2R)-2-(4,5-diphenyloxazol-2-yl)-1-cyclopentyl]methyl}benzamide, N-(2,2-diphenylethyl)-3-{[(1S,2R)-2-(4,5-diphenyl-oxazol-2-yl)-1-cyclopentyl]methyl}benzamide, N-benzyloxy-3-{[(1S)-2-(4,5-diphenyloxazol-2-yl)-2-cyclohexen-1-yl]methyl} benzamide or N-benzylsulfonyl-3-{[(1S)-2-(4,5-diphenyloxazol-2-yl)-2-cyclohexen-1-yl]methyl}benzamide, and the most preferred EP4 receptor antagonists useful for the present invention is N-benzylsulfonyl-3-{[(1S)-2-(4,5-diphenyloxazol-2-yl)-2-cyclohexen-1-yl]methyl}benzamide (FR233074) as represented by the following formula:

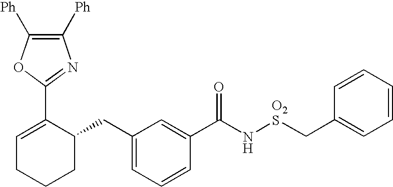

or pharmaceutically acceptable salts thereof.

Renin-angiotensin system inhibitors useful for present invention include a variety of angiotensin converting enzyme inhibitors (ACE-I) and angiotensin II receptor blockers (ARB) known in the art.

Angiotensin converting enzyme inhibitors (ACE-I) useful for present invention include, for example, captopril, lisinopril, temocapril, enalapril, imidapril, quinapril, cilazapril, delapril, trandolapril, ramipril, fosinopril, perindopril, benazepril and alacepril or pharmaceutically acceptable salts thereof (see, e.g., U.S. Pat. No. 4,046,889, U.S. Pat. No. 4,555,502, U.S. Pat. No. 4,699,905, U.S. Pat. No. 4,374,829, U.S. Pat. No. 4,508,727, U.S. Pat. No. 4,761,479, U.S. Pat. No. 4,512,924, U.S. Pat. No. 4,385,051, U.S. Pat. No. 5,256,687, U.S. Pat. No. 4,587,258, U.S. Pat. No. 4,337,201, U.S. Pat. No. 4,508,729, U.S. Pat. No. 4,410,520, and U.S. Pat. No. 4,248,883, which are incorporated by reference in the present specification). Specifically preferable ACE-I useful for present invention is captopril.

Angiotensin II receptor blockers (ARB) useful for present invention include, for example, losartan, candesartan, telmisartan, olmesartan, eprosartan, irbesartan and valsartan, or pharmaceutically acceptable salts thereof (see, e.g., U.S. Pat. No. 5,138,069, U.S. Pat. No. 5,354,766, U.S. Pat. No. 5,591,762, U.S. Pat. No. 5,616,599, U.S. Pat. No. 5,185,351, WO9114679 and EP433983, which are incorporated by reference in the present specification). Specifically preferable ARB useful for present invention is losartan.

Renal diseases treated by the combination of the present invention include, but are not limited to, renal failure (chronic and acute), nephritis, glomerulonephritis, glomerulosclerosis, nephropathy, nephrosclerosis, renal fibrosis, diabetic nephropathy, nephrotic syndrome, and the like. More specifically, renal diseases treated by the combination of the present invention are chronic renal failure and nephritis.

The term “increasing an effect of” means that combination of EP4 receptor antagonist and renin-angiotensin system inhibitor, can more effectively improve renal disease of a patient than EP4 receptor antagonist or renin-angiotensin system inhibitor alone does.

Prostaglandin E2 receptor antagonist and renin-angiotensin system inhibitor used in the present invention may orally or parenterally administered, simultaneously, separately or sequentially, in combination with physiologically acceptable carriers.

Prostaglandin E2 receptor antagonist and renin-angiotensin system inhibitor used in the present invention can be used in the form of a pharmaceutical preparation, for example, in solid, semisolid or liquid form (e.g., tablet, pellet, troche, capsule, suppository, cream, ointment, aerosol, powder, solution, emulsion, suspension etc.), which contains the object compounds or a pharmaceutically acceptable salt thereof as an active ingredient, suitable for rectal, pulmonary (nasal or buccal inhalation), nasal, ocular, external (topical), oral or parenteral (including subcutaneous, intravenous and intramuscular) administrations or insufflations.

Preparation of prostaglandin E2 receptor antagonist and renin-angiotensin system inhibitor used in the present invention can contain various organic or inorganic carrier materials, which are conventionally used for pharmaceutical purpose, such as excipient (e.g., sucrose, starch, mannit, sorbit, lactose, glucose, cellulose, talc, calcium phosphate, calcium carbonate, etc.), binding agent (e.g., cellulose, methyl cellulose, hydroxypropylcellulose, polypropylpyrrolidone, gelatin, gum arabic, polyethyleneglycol, sucrose, starch, etc.), disintegrator (e.g., starch, carboxymethyl cellulose, calcium salt of carboxymethyl cellulose, hydroxypropylstarch, sodium glycol-starch, sodium bicarbonate, calcium phosphate, calcium citrate, etc.), lubricant (e.g., magnesium stearate, talc, sodium laurylsulfate, etc.), flavoring agent (e.g., citric acid, mentol, glycine, orange powders, etc.), preservative (e.g., sodium benzoate, sodium bisulfite, methylparaben, propylparaben, etc.), stabilizer (e.g., citric acid, sodium citrate, acetic acid, etc.), suspending agent (e.g., methyl cellulose, polyvinylpyrrolidone, aluminum stearate, etc.), dispersing agent, aqueous diluting agent (e.g., water), base wax (e.g., cacao butter, polyethyleneglycol, white petrolatum, etc.).

The effective ingredient may usually be administered with a unit dose of 0.01 mg/kg to 100 mg/kg, 1 to 4 times a day. The therapeutically effective dose of ingredient can be suitably determined by practitioner on the basis of age, body weight and condition of patient as well as administration mode.

The following example illustrates the treating renal disease using the combination of prostaglandin E2 receptor antagonist and renin-angiotensin system inhibitor and as such is not to be considered as limiting the present invention in the appended claims.

### EXAMPLE

### Combination Effect of Captopril (ACE-I) and FR233074 (EP4 Antagonist) on Rat Uninephrectomized Anti-Thy-1 Nephritis Model

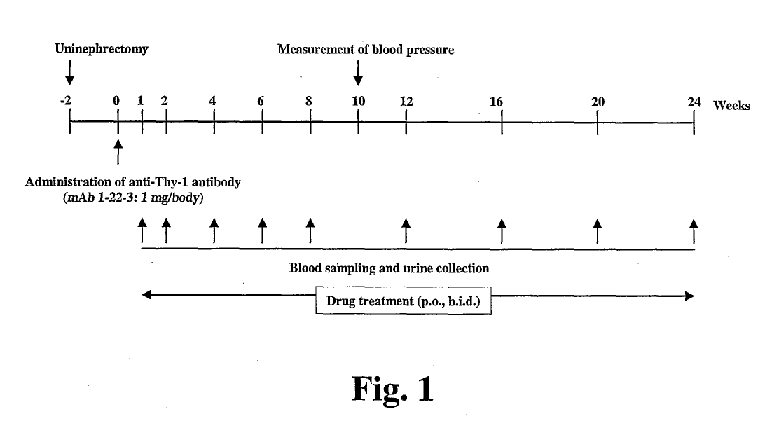

### Method (FIG. 1)

Male Wistar rats (6-weeks old) were used for the example. These Wistar rats were anesthetized by administration of pentobarbital and subjected to unilateral nephrectomy. Two weeks later, these rats were injected with anti-Thy-1 antibody (gift of Department of Cell Biology, Institute of Nephrology, Niigata University Graduate School of Medical and Dental Sciences, Japan) from tail vein of them to induce anti-Thy-1 nephritis. Normal group were subjected to only ventrotomy without unilateral nephrectomy and injected with saline instead of anti-Thy-1 antibody.

One week after administration of the antibody, the rats were weighted, blood of the rats were collected (0.55 ml from subclavian vein), and 24-h urine collections were performed. Then, these rats were divided into the groups based on body weight, renal function parameters (BUN (blood urea nitrogen) and serum creatinine) and urinary excretion of protein.

The agents were administered to the rats for 23 weeks from the day of the grouping. 2, 4, 6, 8, 12, 16, 20 and 24 weeks after administration of the antibody (at 24 weeks after administration of the antibody, the administration of the agents was finished), the rats were weighted, blood of the rats were collected, 24-h urine collections were performed, and then, body weight, renal function parameters and urinary excretion of protein of the rats were determined. Systemic blood pressure of each rat was also measured at 10 weeks after administration of the antibody.

### Results

### 1. Effect on Body Weight

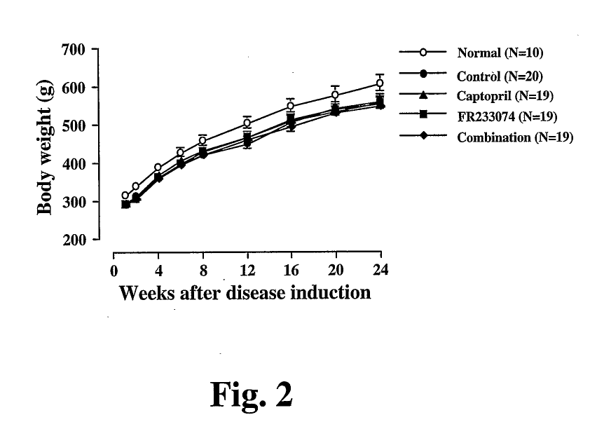

There were no effects of administration of the agents on body weight gain. (FIG. 2)

### 2. Effect on Urinary Excretion of Protein

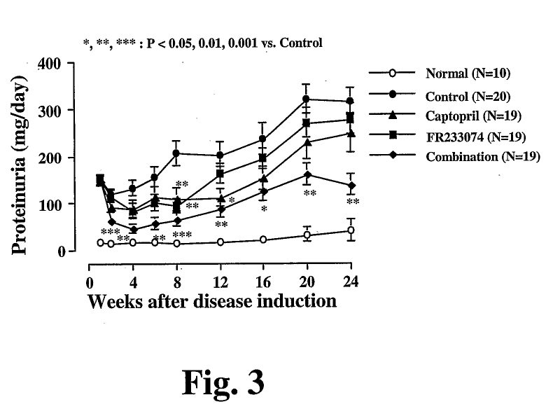

Urinary excretion of protein in control group increased to nine folds of that in normal group on one week after injection of anti-Thy-1 antibody (i.e., disease induction), and continued to increase for 24 weeks (FIG. 3).

On the other hand, in the group administered with captopril alone (30 mg/kg/day) and in the group administered with FR233074 alone (20 mg/kg/day), increases in urinary excretion of protein were suppressed. Furthermore, in the group administered with combination of captopril and FR233074, an increase in urinary excretion of protein was suppressed more strongly, suggesting additive effect of the combination (FIG. 3).

### 3. Effect on Renal Function Parameters

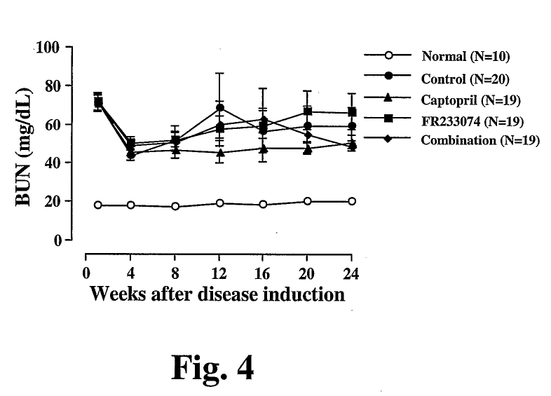

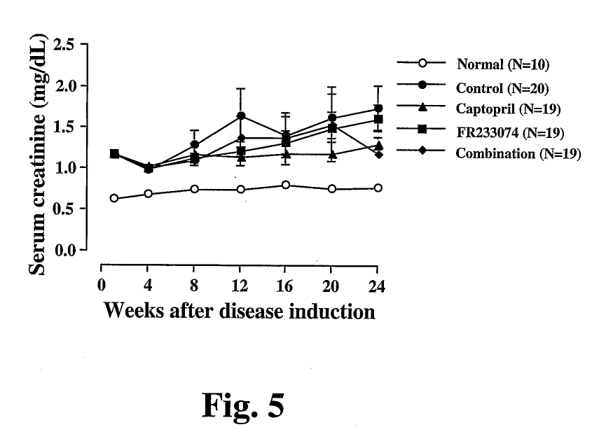

Each group administered with the agent was improved in renal function parameters (BUN and serum creatinine) (FIGS. 4 and 5).

4. Percentage of Rats which Reached to Endpoint

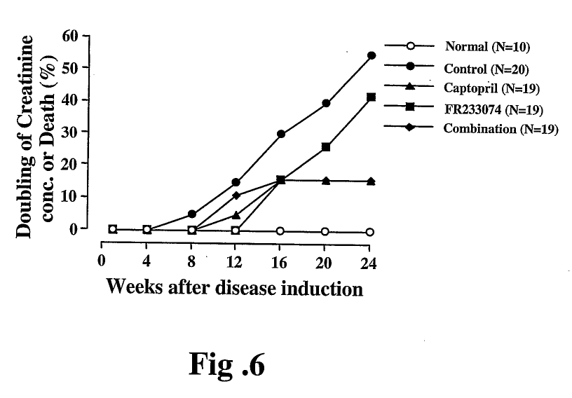

In the control group, rats which reached to endpoint (doubling of serum creatinine concentration or death) appeared on 8 weeks after disease induction and the number of such rats increased thereafter (FIG. 6).

On the other hand, in each group administered with the agent, an increase of rats which reached to endpoint was suppressed, however, there was no significant difference between the group administered with captopril or FR233074 alone and the group administered with the combination (FIG. 6).

### 5. Effect on the Rate of Decline in Renal Function

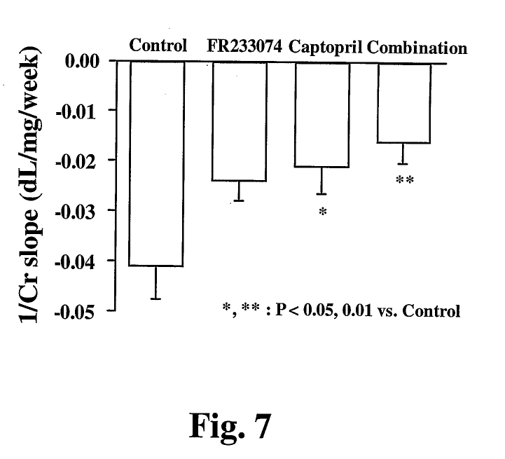

The rate of decline in renal function (1/Cr slope) was decreased in the group administered with captopril or FR233074 alone than the control group. Furthermore, the rate of decline in renal function was more effectively decreased in the group administered with the combination than the group administered with each agent alone (FIG. 7).

### 6. Prediction of Time Reaching Renal Death

An averaged value of 1/Cr in each group at one week after disease induction was plotted, and drew down linearly, based on an averaged value of 1/Cr slope in each group. A time point for 1/Cr=0.1 was calculated, and defined as time reaching renal death (time point for 1/Cr=0.1 is one of standards for introducing dialytic therapy in clinical practice).

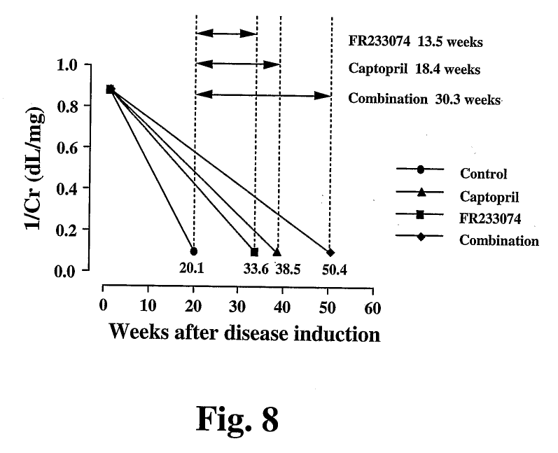

Time from the disease induction to the renal death was prolonged in the group administered with captopril or FR233074 alone compared with the control group. Furthermore, the time was prolonged in the group administered with the combination compared with the group administered with each agent alone (FIG. 8).

### 7. Effect on Systemic Blood Pressure

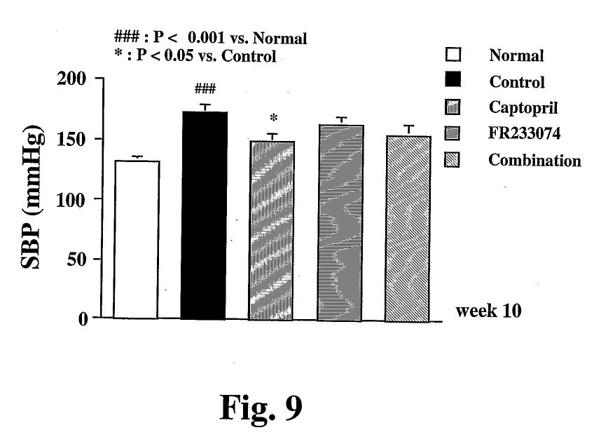

An elevation in systolic blood pressure appeared in the control group. An elevation in systolic blood pressure was suppressed in the group administered with captopril alone or the combination compared with the control group. However, there was no significant effect on an elevation in systolic blood pressure in the group administered with FR233074 alone (FIG. 9).

### 8. Effect on Kidney Weight

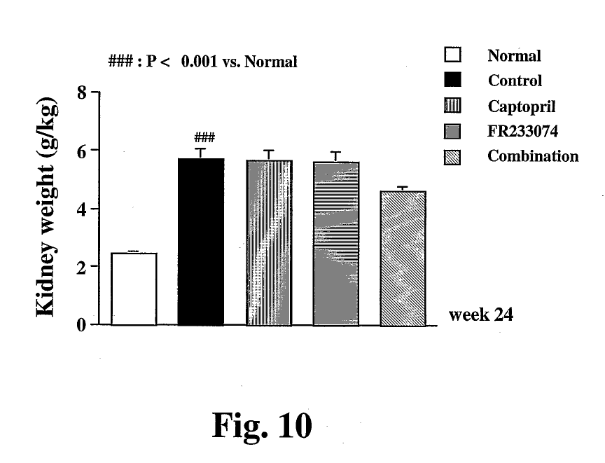

An increase in kidney weight appeared in the control group. On the other hand, it was suppressed in the group administered with the combination (FIG. 10).

### 9. Effect on the Number of Erythrocyte

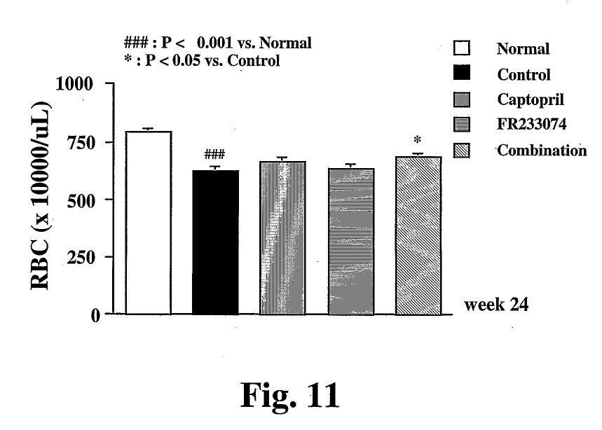

The number of erythrocyte was decreased in the control group due to renal anemia. On the other hand, decrease of erythrocyte was suppressed in the group administered with combination (FIG. 11).

### 10. Histological Observation

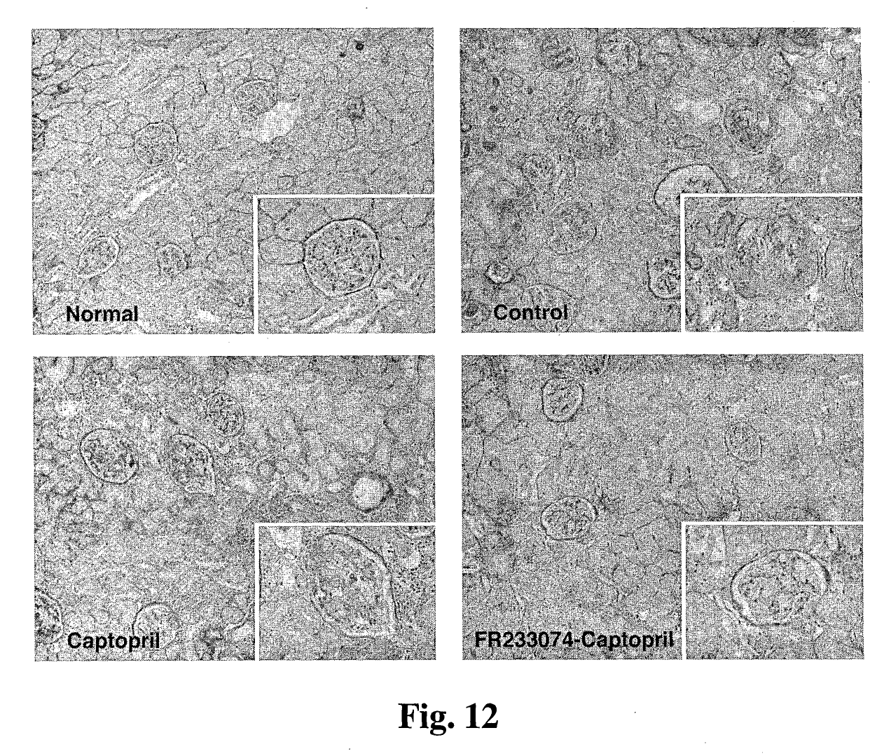

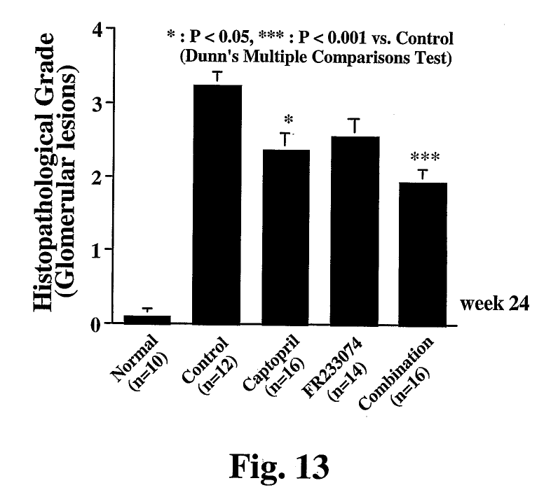

Severe glomerular and tubulointerstitial injury were observed in the control group. However, the extent of damage was reduced in the group administered with captopril or FR233074 alone, and further less damage was observed in the group administered with the combination (FIG. 12-13).

### CONCLUSION

As known from the above result, renin-angiotensin system inhibitor or EP4 receptor antagonist could suppress an increase in urinary excretion of protein (a renal function parameter) and the rate of decline in renal function (an indicator for aggravation of renal injury), and the combination of both agents has additive effect on the suppression of them. Furthermore, the difference in the effects of the agents on systemic blood pressure suggests that each agent can suppress the progression of renal injury by a different mechanism.

1. A method for treating renal disease, comprising administering prostaglandin E2 receptor antagonist and renin-angiotensin system inhibitor to a patient suffering from said disease.

2. The method of claim 1, wherein the prostaglandin E2 receptor antagonist and the renin-angiotensin system inhibitor are administered to the patient simultaneously, separately or sequentially.

3. A method for increasing an effect of renin-angiotensin system inhibitor for treating renal disease, comprising administering prostaglandin E2 receptor antagonist to a patient suffering from said disease.

4. A method for increasing an effect of prostaglandin E2 receptor antagonist for treating renal disease, comprising administering renin-angiotensin system inhibitor to a patient suffering from said disease.

5. The method of claim 1, wherein the prostaglandin E2 receptor antagonist is a prostaglandin E2 receptor EP4 antagonist.

6. A pharmaceutical composition for treating renal disease, comprising prostaglandin E2 receptor antagonist and renin-angiotensin system inhibitor.

7. A pharmaceutical composition for increasing an effect of renin-angiotensin system inhibitor for treating renal disease, comprising prostaglandin E2 receptor antagonist.

8. A pharmaceutical composition for increasing an effect of prostaglandin E2 receptor antagonist for treating renal disease, comprising renin-angiotensin system inhibitor.

9. The pharmaceutical composition of claim 6, wherein the prostaglandin E2 receptor antagonist is a prostaglandin E2 receptor EP4 antagonist.

10. A method of using prostaglandin E2 receptor antagonist and renin-angiotensin system inhibitor for manufacturing a medicament for treating renal disease.

11. A method of using prostaglandin E2 receptor antagonist for manufacturing a medicament for increasing an effect of renin-angiotensin system inhibitor for treating renal disease.

12. A method of using renin-angiotensin system inhibitor for manufacturing a medicament for increasing an effect of prostaglandin E2 receptor antagonist for treating renal disease.

13. The method of claim 10, wherein the prostaglandin E2 receptor antagonist is a prostaglandin E2 receptor EP4 antagonist.

14. An article of manufacture, comprising packaging material and prostaglandin E2 receptor antagonist within the packaging material, wherein said packaging material comprises a label or a written material which indicates that the prostaglandin E2 receptor antagonist can be used for increasing an effect of renin-angiotensin system inhibitor for treating renal disease.

15. An article of manufacture, comprising packaging material and renin-angiotensin system inhibitor within the packaging material, wherein said packaging material comprises a label or a written material which indicates that the renin-angiotensin system inhibitor can be used for increasing an effect of prostaglandin E2 receptor antagonist for treating renal disease.

16. The article of manufacture of claim 14, wherein the prostaglandin E2 receptor antagonist is a prostaglandin E2 receptor EP4 antagonist.
